(19) United States
(12) Patent Application Publication
(10) Pub. No.: US 2005/0142565 A1
(43) Pub. Date: Jun. 30, 2005

(54) Nucleic acid purification chip

(75) Inventors: Victor Samper, Singapore (SG); Ji Hongmiao, Singapore (SG); Chen Yu, Singapore (SG); Heng Chew Kiat, Singapore (SG); Lim Tit Meng, Singapore (SG)

Correspondence Address: Winstead Sechrest & Minick P.C., P.O. Box 50784, Dallas, TX, 75201, US

(73) Assignees: Agency for Science, Technology and Research, Singapore (SG); National University of Singapore, Singapore (SG)

(21) Appl. No.: 10/818,532

(22) Filed: Apr. 5, 2004

Related U.S. Application Data
(60) Provisional application No. 60/533,297, filed on Dec. 30, 2003.

(51) Int. Cl. C12Q 1/68; C12M 1/34

(52) U.S. Cl. 435/6; 435/287.2

(57) ABSTRACT

The present invention provides for a novel system of extracting and purifying nucleic acids (DNA, RNA, etc.) from cellular material like blood. Such a system of extraction and purification relies on novel monolithic microfluidic devices and methods of using these devices. Such devices comprise numerous components, monolithically-incorporated on an single chip, and further comprising novel nucleic acid binding materials. The present invention is also directed to method of preparing such novel nucleic binding materials.

### CROSS REFERENCE TO RELATED APPLICATIONS

The present application claims priority to the following U.S. Provisional Patent Application, Ser. No. 60/533,297, filed Dec. 30, 2003.

### TECHNICAL FIELD

The present invention relates in general to biotechnology, and in particular, to chip-based microfluidic methods and devices for extracting and purifying nucleic acid.

### BACKGROUND INFORMATION

Genomics has wide application for areas such as criminal analysis, clinical diagnosis, etc. It is employed in such diverse fields as agriculture, health care, environmental monitoring, and pharmacology research. In most cases, genomic DNA is obtained from white blood cells that come from human blood. Processes used to obtain such DNA usually require the isolation of nucleic acids from their respective biological sources. In the case of human DNA purification from blood, the DNA is initially confined inside white blood cells. To extract and purify this DNA, the cell membrane must be opened (lysed) using one or more of a variety of different methods that include chemical, osmotic, thermal, electrical, and physical means.

Current techniques for obtaining such DNA from blood at a hospital or laboratory are still quite arduous and require numerous, often manual, steps. The pervious step is the lysis of the blood cells, wherein the cell is broken open and its nucleic acids are released into the solution and corresponding area available for purification. The process of cell membrane rupture and release of the nucleus contents means that other biological molecules will also be present in the solution. Some of these molecules, such as proteins and metal complexes (for example, hemoglobin), bind with the nucleic acid in an undesired manner and otherwise interfere with typical subsequent processing steps such as amplification by PCR (polymerase chain reaction). Consequently, a step to separate the DNA from the debris material is needed. After that, the requirement of any DNA purification system is that the nucleic acid must be isolated while these inhibitors are being washed away. All of these steps are generally manual operations requiring a large amount of time. Thus, it is important that some method capable of doing these operations automatically, using less sample, be developed.

It would be useful to produce a microfluidic chip capable of handling and processing microliter, μl, (or smaller) volumes of whole human blood to provide purified genomic DNA as the output in an automated fashion. Optimally, this DNA sample preparation microfluidic chip is an integrated device with a micromixer, microfilter, microreactor, etc. The output should have similar yield and purity as conventional macroscopic systems in use today. The current trends in techniques for molecular biology require that the test procedures retain good yield results whilst reducing the assay time, reagent volume, and cost. Furthermore, increasing levels of automation are more common. There is also a demand for miniaturization so that the tests need not be confined to a central laboratory, but can be portable enough for field applications or point-of-care testing.

For the chip's design, individual filter, valve, mixer, and reactor components have been published many times within the past 10 years, but these designs for the different components have specific applications precluding their combination for general purpose use. Integration of all the components required to realize DNA sample preparation/purification remains a major challenge. While the integration of multiple components on a polymer substrate has been previously published, these generally are used for microarray, PCR systems. The only known example of a full microfluidic solution technique for DNA sample preparation/purification from blood has been published by Kim et al. See Kim et al., “A Disposable DNA Sample Preparation Microfluidic Chip for Nucleic Acid Probe Assay,” IEEE-MEMS 2002, pp. 133-136. This integrates a silicon filter with a glass reactor on a PDMS substrate (substrate includes a mixer). The arrangement reported by Kim et al. is a variation of micro-machined component integration on a polymeric substrate. The distinct components are the binder (fabricated from glass), and the filter (fabricated from silicon, nickel, and PDMS). The connecting substrate differs from a conventional interconnect substrate since the interconnects form a micromixer.

A typical protocol currently used for nucleic acid purification involves a number of steps whereby various reagents are added to the sample, and the sample is centrifuged to separate precipitated components from the solution. Such a procedure is quite involved and, in many cases, is still done manually. Since nucleic acid is known to selectively bind to some surfaces, a great deal of research is focused on these types of interactions. A number of surfaces suitable for nucleic acid binding have been found and such binding techniques utilize silica bead binding, tethered antibodies, silanes, synthesized nucleic acids, polylysine, poly-T-DNA, some acids and bases.

Several methods and devices have recently been developed which attempt to improve the genomic analysis system within microscale devices. U.S. Pat. No. 6,379,929, discloses methods and compositions for isothermal amplification of nucleic acids in a microfabricated substrate. Methods and compositions for the analysis of isothermally-amplified nucleic acids in a microfabricated substrate are disclosed as well. The microfabricated substrates and isothermal amplification and detection methods provided are envisioned for use in various diagnostic methods, particularly those connected with diseases characterized by altered gene sequences or gene expression. However, these focus solely on amplification of nucleic acid, which is not considered DNA extraction and purification.

U.S. Pat. No. 6,368,871 describes a device and method for the manipulation of materials (e.g., particles, cells, macromolecules, such as proteins, nucleic acids or other moieties) in a fluid sample. The device comprises a substrate having a plurality of microstructures (pillars) and an insulator film on the structures. Application of a voltage to the structures induces separation of materials in the sample. The device and method are useful for a wide variety of applications such as dielectrophoresis (DEP) or the separation of a target material from other material in a fluid sample. Such techniques use the pillars' structure, to which a voltage has been applied, to facilitate mixing.

U.S. Pat. No. 6,168,948 provides for a miniaturized integrated nucleic acid diagnostic device and system which includes a nucleic acid extraction zone including nucleic acid binding sites. The miniaturized nucleic acid extraction and sample refinement device disclosed in this patent comprises a porous flow-through deformable plug for binding nucleic acid, or structures having binding sites for sample within a chamber. This plug is formed or added after the microfluidic channel formation, making it an assembly or in situ synthesis process. For this reason, it cannot be described as being monolithic or as having the advantages of batch fabrication that arise from a monolithic design.

### SUMMARY OF THE INVENTION

The present invention is directed to devices and methods for the extraction and purification of DNA from cells. Such devices and methods provide for the systematic removal and separation of nucleic acids from cellular material. Typically, the cellular material is obtained from blood (i.e., white blood cells).

In some embodiments, the present invention is directed to microfluidic devices. The devices of the present invention are monolithic in their design and construction. In some embodiments, the microfluidic devices comprise a silicon substrate, inlets for introducing microliter quantities and more of material, mixers, reaction chambers, nucleic acid binding material, and outlets for removing material from the device.

The binding material selectively binds to nucleic acid. In some embodiments, the binding material is prepared by first treating a silicon substrate with a thermal oxide process, and then plasma etching the silicon oxide surface with a plasma etchant process (plasma treatment). In additional or other embodiments, the binding material of the present invention is produced by depositing silane-based silicon oxide on a substrate using a plasma-enhanced chemical vapor deposition (PECVD) process.

In some embodiments, the present invention is directed to monolithic microfluidic devices made by novel processes. Such devices provide for the extraction and purification of DNA from cellular material, but are constructed in novel, cost-efficient ways and comprise novel binding material produced in a novel and efficient manner.

In some embodiments, the present invention is directed to methods for processing nucleic acids. In such embodiments, cellular material is ruptured (lysed) to release contents, and the nucleic acid portion of those contents is isolated. Such methods typically employ chemical techniques to lyse the cellular material. Isolation of the nucleic acid content is accomplished, in part, via selective binding to a binding material under controlled conditions, wherein the binding material is a novel binding material of the present invention.

The present invention differs from that of Kim et al. in that its design is monolithic and on silicon. This means that there is no assembly of multiple components. It also means that dead volumes are smaller by maintaining the majority of the fluid flow in microchannels within the plane of the substrate (low dead volume), with only the inlet and outlet streams making the transition to flows that are normal to the plane of the substrate (high dead volume). Monolithic integration on silicon also provides the possibility of thermal isolation between components, resulting in reduced steady state power consumption at elevated temperatures, uniform temperature profiles within reactors, rapid changing of the system temperature by changing the heat sink connected to the substrate, rapid thermal cycling, and systems consisting of different simultaneous temperature zones.

The foregoing has outlined rather broadly the features of the present invention in order that the detailed description of the invention that follows may be better understood. Additional features and advantages of the invention will be described hereinafter which form the subject of the claims of the invention.

### BRIEF DESCRIPTION OF THE DRAWINGS

For a more complete understanding of the present invention, and the advantages thereof, reference is now made to the following descriptions taken in conjunction with the accompanying drawings, in which:

FIGS. 1A and B are system diagrams illustrating two embodiments of the present invention, wherein the difference between the two resides in the presence (B) or absence (A) of a filter component;

FIG. 2 is a flow diagram of a generalized method of extracting and purifying nucleic acid according to the present invention;

FIG. 3 illustrates the binding and elution mechanism whereby nucleic acid binds to the binding material under high salt conditions (A), and is eluted under low salt conditions (B);

FIG. 4 illustrates, schematically, a plane cross-sectional view of an embodiment of the present invention, which was designed with a silicon-glass bonded structure;

FIG. 5 illustrates a chip according to some embodiments of the present invention, comprising numerous components;

FIGS. 6A and B illustrate gel electrophoresis plates wherein the presence (or absence) of bands in a given lane is reflective of the binding ability for one of four differently-prepared binding materials;

FIG. 7 illustrates a relationship between temperature and binding efficiency, according to embodiments of the present invention; and

FIG. 8 illustrates the nucleic acid elution efficiency, at a variety of temperatures, for devices comprising binding materials made by a variety of methods and/or treatments.

### DETAILED DESCRIPTION

The present invention is directed to devices and methods for the extraction and purification of DNA (or other nucleic acid) from cells. Collectively, or in part, these devices and methods can form systems that provide for the extraction and purification of such nucleic acids.

The following definitions are provided for a better understanding of the present invention.

A “nucleoside” is a purine or pyrimidine base linked glycosidically to ribose or deoxyribose. A “nucleotide” is a phosphate ester of a nucleoside. An oligonucleotide is a linear “sequence” of up to 20 nucleotides, or “mers,” joined by phosphodiester bonds. A “nucleic acid” is a linear polymer of nucleotides (as in an oligomer, but longer), linked by 3′,5′ phosphodiester linkages. In “DNA,” deoxyribonucleic acid, the sugar group is deoxyribose, and the bases of the nucleotides are adenine (A), guanine (G), thymine (T), and cytosine (C). “RNA,” ribonucleic acid, has ribose as the sugar group, and the same nucleotide bases, except uracil (U) replaces thymine. A single strand of DNA has a “sequence” of bases A,G, T, and C. When forming a DNA double-helix, for example, this secondary structure is held together by hydrogen bonds between bases on the neighboring strands. Note that in such base pairing, A always bonds to T and C always bonds to G.

“Genomic DNA” is the DNA which is found in the organism's “genome” (i.e., all the genetic material in the chromosomes of a particular organism); its size is generally given as its total number of base pairs and is passed on to offspring as information necessary for survival. The phrase is used to distinguish between other types of DNA, such as found within plasmids. “Genomics” refers to the study of genomes, which includes genome mapping, gene sequencing and gene function.

“Gene sequencing” refers to the determination of the relative order of base pairs, whether in a fragment of DNA, a gene, a chromosome, or an entire genome. Critical to many methods of gene sequencing are electrophoretic separation techniques such as “gel electrophoresis,” “capillary electrophoresis,” and “disc electrophoresis.”

“PCR,” polymerase chain reaction, is a system for in vitro amplification of DNA wherein two synthetic oligonucleotide primers, which are complementary to two regions of the target DNA (one for each strand) to be amplified, are added to the target DNA in the presence of excess deoxynucleotides and Taq polymerase, a heat-stable DNA polymerase. In a series of temperature cycles, the DNA is repeatedly denatured, annealed to the primers, and a daughter strand extended from the primers. As the daughter strands act as templates in subsequent cycles, amplification occurs in an exponential fashion.

“Reactive ion etching” (RIE) or “deep reactive ion etching” (DRIE) refers to techniques whereby radio frequency (RF) or microwave radiation is coupled into a low pressure gas to ionize the gas producing disassociation of the gas molecules into more reactive specie, and the substrate being etched (typically silicon based) is biased to induce ion bombardment. Compounds containing carbon (C) and halogens such as, fluorine (F), chlorine (Cl), or Bromine (Br) are typically used as gases. When the compounds dissociate in the plasma, both highly reactive halogen atoms or halogen compounds, and polymers that may deposit on the substrate blocking the highly reactive species are generated. Ions accelerated towards the substrate being etched by the applied or induced bias remove polymers on substrate surfaces oriented normal to the direction of ion motion, polymers coat substrate surfaces that are oriented parallel to the ion motion and block etching of those surfaces. Ion bombardment may also activate or accelerate chemical etching reactions. RIE therefore has the capability to etch surfaces normal to the direction of ion motion at a higher relative rate and surfaces parallel to the ion motion at a lower relative rate resulting in anisotropic etching.

“Microelectromechanical systems” (MEMS) result from the integration of micromechanical structures (containing moving parts) with microelectronics.

A “thermal oxide process,” according to the present invention, involves heating silicon [wafer] to temperatures between 600° C. and 1250° C. in the presence of oxygen (O₂) and/or steam (H₂O). These elevated temperatures enhance the diffusion of the oxidant and result in oxides significantly thicker than the 2 nm native oxide that results from silicon oxidation in air at room temperature.

“Chemical vapor deposition” (CVD) refers to material deposition from gas-phase chemical precursors.

“Monolithic,” or “monolithic integration,” according to the present invention, means that all components have been designed with a common technology, fabricated simultaneously on a common substrate, and direct fluid flow in the plane of a substrate wafer surface.

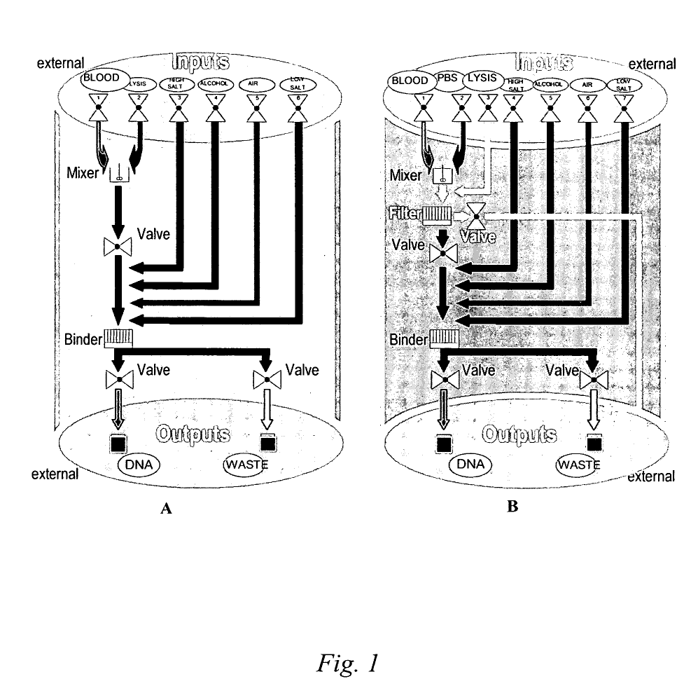

Now referring to FIG. 1, the present invention can be viewed in system diagrammatic terms. Shown in FIG. 1A, various inputs (blood, lysing agent, high salt solution, alcohol, air, and low salt solution) are introduced into a microfluidic device of the present invention by way of valves. Initially, blood and lysing agent are introduced, via valves, into a mixer or mixing chamber. Upon mixing, the lysing agent ruptures the cell membranes of the white blood cells (WBC) and (red blood cells (RBC) within the blood so as to release the nucleic acid material contained within the WBC. After lysing, a valve then directs the nucleic acid (along with other waste material from the blood and blood cells) to a binding chamber, comprising a binder comprised of binding material, under conditions of high salt. The flow of high salt solution ensures that the nucleic acid selectively binds to the binding material, but the waste material passes through and out as waste. With the nucleic acid still bound to the binding material, the binder is rinsed with alcohol (e.g., ethanol), then dried with air (force convection), before it is finally eluted with a low-salt solution. Accordingly, blood is contacted with the lysing agent and both the WBC and RBC are lysed together inside the mixer. The cellular contents are released and DNA (from the WBC) binds to the binder. In this embodiment, the cells can be lysed together using just one mixer prior to binding to achieve the procedure of lysis, binding, and elution.

Alternatively, the system can employ a filter capable of trapping white blood cells, but permitting red blood cells and other material to pass prior to introduction of the lysing agent. Such an embodiments is shown in FIG. 1B, wherein blood is introduced and mixed together with a phosphate buffer solution (PBS) prior to being passed through a filter. After rinsing away the RBC and other material, the WBC are lysed and passed into the binding chamber for further purification, as put forth above. This embodiment thus allows for crude purification prior to exposure to the binding material.

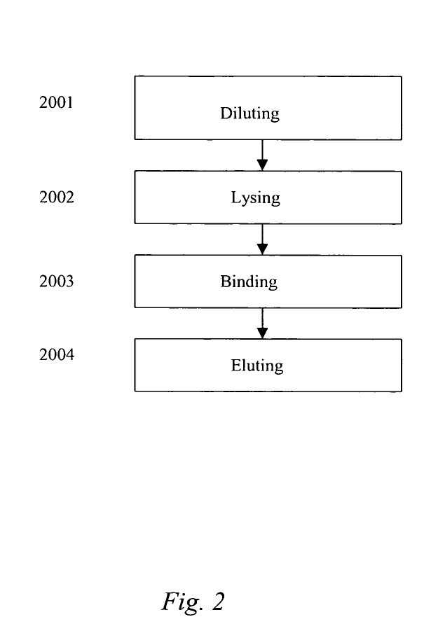

Viewed in a different manner, the present invention is a method for extracting and purifying nucleic acid from cellular material. Such methods generally comprise a series of steps. Referring to FIG. 2, step 2001 is a step of diluting the blood so as to decrease viscosity and render the mixture more amenable to flow in the microchannel and microchamber regions of the devices to be described later. Dilution can be realized with the addition of a phosphate buffered solution (PBS) or other similar solution. Step 2002 is a step of lysing whereby a lysing agent is added to the diluted blood solution to rupture cell membranes and release nucleic acid material from the WBC. Note that DNA is only found in the WBC in the blood, as RBC have no nucleus. Such lysing agents are typically chemical lysing agents (chemical lysis), but other types of lysis could be employed as well such as ultrasonic lysing, thermal lysing, electrolysis, and mechanical rupture of the cell membrane (known as mechanical lysing). Step 2003 is a step of binding the released nucleic acid to a binding material under condition of high salt content. This is accomplished via the careful control of the salt content (i.e., concentration) within the solution, and by providing a specially treated binding surface (binding material). The released nucleic acid is directed to the binding material/chamber via a combination of fluid flow and valves. Step 2004, the step of eluting, is realized when the salt content condition is changed to one of low salt content or water.

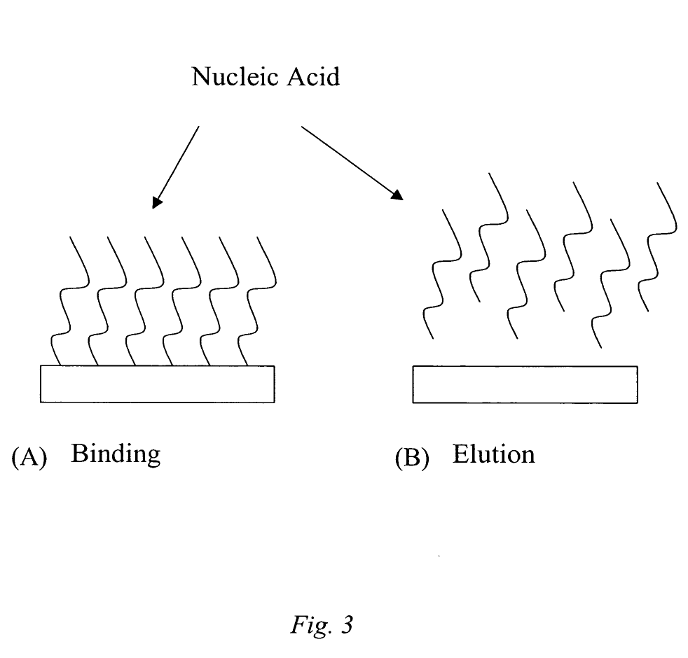

FIG. 3 illustrates the process of binding to binding material or substrate under high salt conditions and eluting, involving a de-binding process, under low salt conditions. Additional steps, such as filtering, washing, rinsing and drying can also be added. Such washing steps can include a high salt wash to make the binding stronger, an alcohol wash to clear the debris or other waste material, and an air-dry to clear the alcohol. Such methods utilize novel devices that represent embodiments of the present invention in their own right.

In some embodiments, the present invention is directed to microfluidic devices. In some embodiments, the microfluidic devices comprise a silicon substrate, inlets for introducing microliter quantities of material, mixers, reaction chambers, nucleic acid binding material, and outlets for removing material from the device. Such devices typically comprise components that have been monolithically integrated.

The binding material selectively binds to nucleic acid. In some embodiments, the binding material is prepared by first treating a silicon substrate with a thermal oxide process, and then plasma etching the silicon oxide surface with a plasma etchant process. In additional or other embodiments, the binding material of the present invention is produced by depositing silane-based silicon oxide on a substrate using a plasma-enhanced chemical vapor deposition (PECVD) process with or without a subsequent plasma etchant process. In additional or other embodiments, other types of chemical vapor deposition (CVD) processes such as tetra-ethylorthosilicate (TEOS) may be used to deposit silicon oxide, with or without a subsequent plasma etchant process.

In some embodiments, the present invention is directed to microfluidic devices incorporating novel and functional design. Such devices provide for the extraction and purification of DNA from cellular material, but are constructed in novel, cost-efficient ways. Such devices comprise a monolithic design and novel binding materials.

Collectively viewed as a microfluidic sample processing system, the devices and methods of the present invention provide for a monolithic chip designed for nucleic acid preparation (e.g., purification). More specifically, the present invention provides for the extraction and purification of DNA and/or RNA (nucleic acid) from blood (generally human, but also other mammalian and non-mammalian species). For the devices of the present invention, functional microfluidic components are typically integrated on a single substrate. Such components possess functions that include mixing, filtration, binding, and others. While the present invention has been demonstrated successfully for DNA extraction, its use is not limited to DNA extraction and can be used for the purification of any species capable of being selectively bound to the binding agents of the present invention, either directly or indirectly, through an inter-mediating substance or molecule.

Monolithic integration, as employed in the present invention, provides devices in which all components have been designed with a common technology and use fluid flow in the plane of the wafer surface, except for fluid inlets and outlets where flow may be normal to, or in the plane of, the wafer surface. Such monolithic design of the components provides for easier production and operation.

Advantages of the present invention over currently used macroscopic systems are: fully automatic operation, and small size and power consumption for portable applications. Advantages over non-monolithic micro-scale solutions are: ease of assembly and packaging, smaller dead volume, overall size, improved thermal design (due to thermal properties of silicon and geometric micromachining possibilities), the option to add additional sensing functionality since all the components can be readily interfaced to electrical readout connections, and the option to partially or fully automate the system.

Surface condition, surface area, flow profile, chemicals used, pH, and temperature—all have a profound effect on the present invention's ability to extract and purify nucleic acid. Consequently, the methods of the present invention provide for the careful control of such parameters. This is particularly true with respect to the design and operation of the devices of the present invention.

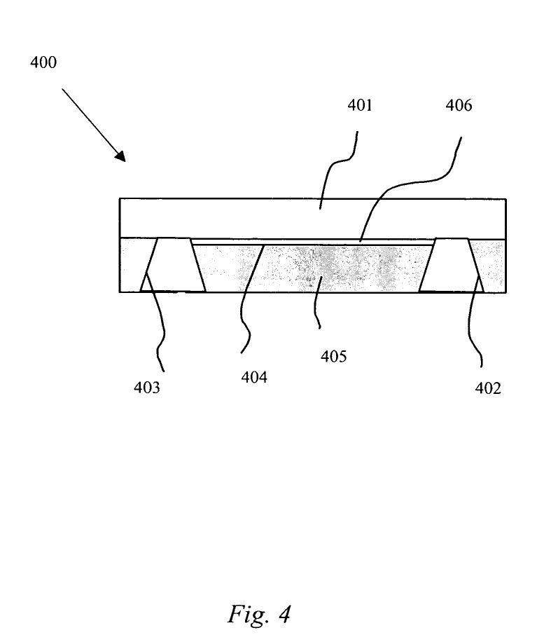

The present invention is also directed toward monolithic microfluidic devices capable of providing for the extraction and purification of nucleic acid from cellular material. FIG. 4 shows the plane view cross-sectional schematic diagram of a device embodiment of the present invention, which was designed with a silicon-glass bonded structure. In this particular embodiment, the device 400 is formed in substrate 405 and covered by glass wafer 401. Cover 401 is transparent to allow optical access to the channels 406. Features 402 and 403 are silicon backside openings that provide the inlet and outlet. Optical access to the device enables optical sensing of the device performance by making such things as the occurrence of blockage, flow rate, flow rate uniformity across the channel width, fluid interfaces, and progression of fluid interfaces through the system, determinable. Optical access to the device also enables optical detection of reaction products by fluorescence, absorbance, or other typical optical techniques that are possible through a glass viewing window.

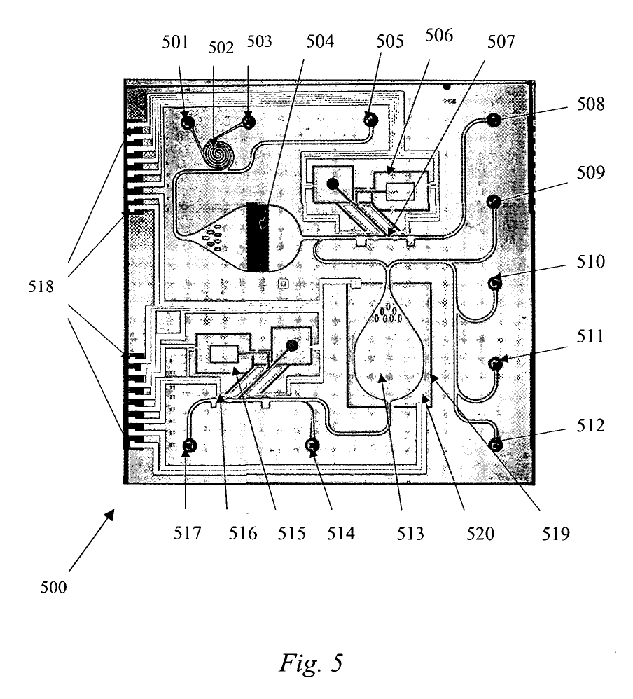

For typical device embodiments, according to the present invention, the reader is directed to FIG. 5 showing device 500. According to FIG. 5, blood and phosphate-buffered saline (PBS) solution can be introduced into mixer 502 by inlets 501 and 503, respectively. The flow then travels directly into a filter 504 which traps WBC, but allows RBC to pass through and go out via outlet 508. Note that filter 504 can be omitted, as previously mentioned in the system description above (see FIG. 1A). After that, a lysis buffer is introduced by inlet 505 and lyses WBC which have been trapped by the filter. After WBC have been lysed, the released DNA will pass through filter 504. At this time, valve 506 will operate to close the channel 507. The DNA with other components and solution will go to the binder 513 and bind to the binder's surface. After the nucleic acid binds to the binder 513, a high salt solution can be pumped through inlet 509 to make binding stronger, and then alcohol is pumped through inlet 510 to wash the binder and make the binder clean save for the nucleic acid inside the binder. After that, force convection is used to dry the binder, especially to dry (i.e., remove) the alcohol, since alcohol will affect the quality of nucleic acid for the post-purification reactions of nucleic acid such as PCR. The last step is to pump a low salt solution at inlet 512 to release the DNA. At this time, the valve 515 will operate to close the channel 516. Then, the DNA will be eluted from the outlet 514. Optionally, thermal isolation trenches 519 and resistive heaters 520 can be added to thermally influence the lysing/binding/elution processes. Additionally, the above-mentioned force convection can be generated via vacuum or by forcing compressed air through the inlet 511 to dry the binder. Compared with traditional natural convection, this method is faster and easier to control.

There is flexibility in the order of treatment and in the location of sample (blood) and/or reagent introduction. In some embodiments, samples and reagents can be introduced into microchannel intersections, directly into reactors, or passed through one or more mixers-depending on the level of mixing required and the flow rates involved.

In some embodiments, the chip is fabricated on a silicon wafer or silicon/glass wafer combination using traditional MEMS fabrication technology. All the components, like mixer, filter, and binder, reside on one chip in order to carry out certain functions required for sample preparation, processing and purification. These components include the channels, inputs, outputs, reactor, mixer, filter, binder, resistive temperature sensors, and resistive heaters. Furthermore, thermal isolation features on the substrate provide for independent thermal operation of components.

An important aspect of the above-mentioned device components is the binder's design. The binder is the component directly responsible for the DNA or RNA's isolation and purification. When the solution which included the nucleic acid and other debris or rubbish passes through the binder, the binder will selectively bind the nucleic acid and let the other material pass through. In a subsequent step, the binder will release the nucleic acid under certain engineered conditions. Thus, the binder's surface is important in such kinds of chips. The present invention can employ one or more of several different designs for this surface which include, but are not limited to, silica beads binding, acids, bases, silanes, polylysine, tethered antibodies, synthesized nucleic acids, and Poly-T DNA. In some exemplary embodiments, depending on the fabrication process, the binder chamber's surface can be generated using a thermal oxide process with subsequent CHF₃ and O₂ plasma etching treatment or, alternatively, plasma-enhanced chemical vapor deposition (PECVD) of silane-based silicon oxide. Such processes generate a surface that is good at nucleic acid binding and elution. Using this kind of surface, there are no extra process steps required for the chip surface modification. The plasma treatment step can be done during the wafer front side nitride stripping process, which is a necessary step for the chip fabrication anyway.

In some embodiments, binder (binding material) is designed with a plasma treated surface and also considers how temperature affects the binding process. In some embodiments, it has a heater and temperature sensor external to the binder in order to provide for different and/or uniform temperature for better binding conditions. Thermo-isolation has also been considered. In this case, applied heat can be localized through thermal isolation features on the substrate allowing different parts of substrate to be at different temperatures.

The microfluidic sample processing system of the present invention integrates all sample preparation processes, like mixing, filtration, binding, elution, together with individual thermal control. It is a generic system which has been demonstrated successfully for DNA extraction, but its use is not limited to DNA extraction. Monolithic integration means all components have been designed with a common technology and use fluid flow in the plane of the wafer surface. Thermal isolation features have been incorporated so that different areas of the chip are thermally independent.

The surface of the binding material is an important factor for the above-mentioned binding efficiency. Another important factor to the binding is surface area. To increase the binding surface area, different methods can be employed. One such method is to introduce some micro-machined features, such as increasing the number of the pillars (microstructures), which increase the vertical area for the binder's surface area. To be sure, the design and arrangement (placement) of such pillars (or other similar microstructures) must consider easier flow patterns, fewer bubbles, a decreased level of clogging, and other related problems. Another method is to make the surface rougher by a chemical method or a physical method in order to increase the surface area. Surface roughening can be used to increase the available surface area for binding on the glass wafer, 401, or the silicon wafer 405. The techniques to roughen the surface of glass include reactive ion etching, plasma etching, and wet etching. In all such cases, the surface roughness can be increased, but a non-uniform process may arise such as that arising from natural micro-masking of the glass surface during etching by bubbles, reaction products, or non-volatile components in the glass. Similar techniques can be employed to increase the surface roughness of silicon. Additionally, silicon surface roughening can be achieved on the side wall of the channel after deep reactive ion etching (DRIE) by tuning the process to increase the natural scalloping that arises in the bosch DRIE process.

Additional advantages of the present invention over currently used commercial extraction kits used for extracting DNA from cells include: decreased amounts of reagents needed (˜2 ml per sample vs. ˜400 ml per sample), smaller blood sample required (˜1 μl per extraction vs. ˜300 μl per extraction), and decreased extraction time (<2 hours per run vs. ˜1 day per run).

The following examples are provided to demonstrate particular embodiments of the present invention. It should be appreciated by those of skill in the art that the methods disclosed in the examples which follows merely represent exemplary embodiments of the present invention. However, those of skill in the art should, in light of the present disclosure, appreciate that many changes can be made in the specific embodiments described and still obtain a like or similar result without departing from the spirit and scope of the present invention.

### EXAMPLE 1

This Example illustrates how a microfluidic nucleic acid purification chip of the present invention can be fabricated. Such a chip may be fabricated by the following steps:

Step 1: Bare silicon wafer is oxidized by thermal oxidation to an oxide thickness of about 0.5 μm. A 0.15 μm-thick layer of low-pressure chemical vapor deposited stoichiometric silicon nitride is then deposited on the silicon oxide.

Step 2: The wafer from the previous step is then masked for DRIE. The mask layer can be photoresist, but this may need to be changed to another material for RIE depths more than about 40 μm. The photoresist is then removed after silicon etching.

Step 3: The channels of the wafer from the previous step are etched on the front side of the silicon wafer using DRIE.

Step 4: Next, the backside of the silicon wafer from the previous step is selectively masked by photoresist, and the openings for the backside fluidic inlets and outlets are etched into the silicon nitride and silicon oxide using reactive ion etching. The photoresist is then removed.

Step 5: The silicon wafer frontside is protected next in a one-sided chuck and the wafer is etched in potassium hydroxide solution until the backside hole reaches the bottom of the features etched on the frontside by DRIE.

Step 6: The silicon nitride on the front side of the wafer is then removed by a plasma etch (CHF₃ and O₂), exposing the silicon oxide below.

Step 7: Next, a layer of about 1 μm-thick Al is sputtered onto the glass wafer.

Step 8: The sputtered Al is then selectively masked by photoresist and etched using a standard phosphoric acid-based aluminum wet etchant, as used by the semiconductor industry.

Step 9: Finally, the photoresist is removed from the glass wafer and the glass wafer is anodically bonded to the front side of the silicon wafer. The glass wafer is aligned to the silicon wafer before bonding.

The resulting two-wafer device, fabricated by the above-described process, comprises microfluidic channels etched on the frontside of the silicon wafer. These are connected to the outside world via holes etched on the backside of the silicon wafer. The fluidic channels vary in size from about 2 μm in width to more than 5 mm in width. Typical channels are about 100 μm wide. Typical filters consist of pillars with about 2-3 μm pillar separation, with the pillars being about 10 μm wide and deep. The channels are closed by the glass capping wafer.

In other embodiments, the holes for connecting the microfluidic structures to the outside world can be made by drilling holes in the glass wafer prior to anodic bonding. In such cases there is no need for the above-mentioned Step 5 wherein the silicon wafer frontside is protected in a one-sided chuck and the wafer is etched in potassium hydroxide solution until the backside hole reaches the bottom of the features etched on the frontside by DRIE. In alternative or other embodiments, the aluminum layer steps (Steps 7 and 8) are not required, as the aluminum layer is used as a heater, temperature sensor, or flow sensor and is not required for all embodiments of the invention. In some or other embodiments, the bond pad connection to the aluminum layer is achieved by opening large regions on the backside of the silicon, large enough for the wire bonding tool to access the bond pads.

Finally, it is worth noting that the reaction chamber and the filter chambers typically have volumes of about 0.4 μl and the mixer has a dead volume of about 0.15 μl. The backside holes are typically 1 mm×1 mm openings on the backside of the wafer, with the characteristic 54° slopes associated with anisotropic wet etching of <100> silicon.

### EXAMPLE 2

This example serves to illustrate the effect of further plasma treatment on the thermal oxide-produced binding material as used in embodiments of the present invention.

In this Example, the purification efficacy of a thermal oxide-generated binding material was evaluated by comparing the eluant of four different binding materials:
Thermal oxide alone.
Thermal oxide+hydrogen peroxide/sulfuric acid (“Piranha,” comprising a 3:1 conc. H₂SO₄:30% H₂O₂) clean.
Thermal oxide+plasma etching.
Thermal oxide+plasma etching+hydrogen peroxide/sulfuric acid (Piranha) clean.
   wherein the plasma treatments comprised a CHF₃+O₂ environment.

The DNA was bound to the respective binding material under the same test conditions for each of the four differently-prepared binders using typical high salt chaotropic conditions, such as 6M guanidine hydrochloride solution, and the material was then rinsed in a clean 6M guanidine hydrochloride solution. The DNA was then eluted under low salt conditions using 1×TE buffer (10 mM Tris-Cl and 1 mM EDTA) and amplified using PCR.

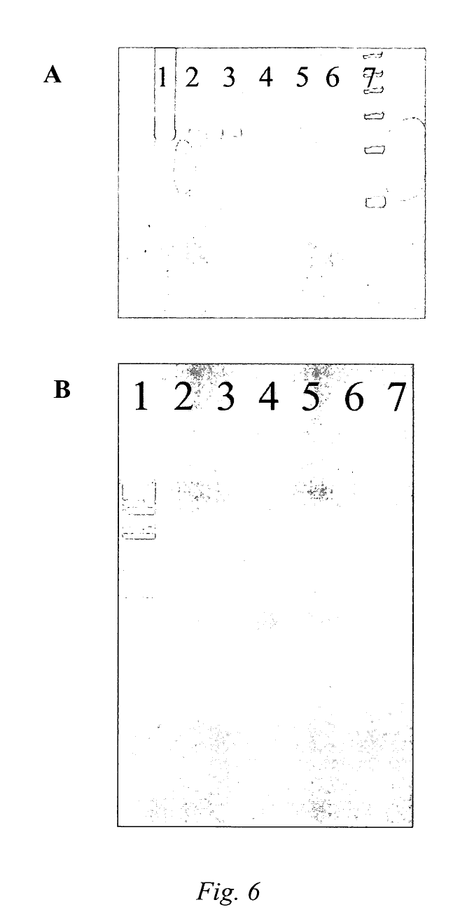

FIG. 6A shows a gel electrophoresis plate of the results after PCR wherein lanes 4 and 5 correspond to eluant from a device with the thermal oxide alone. The results indicate that little or no reversible binding of the nucleic acid occurred under the conditions employed since lanes 4 and 5 correspond to the eluant and no band associated with the DNA fragment under test can be seen. For comparison, the band can be clearly seen in lanes 2 and 3. FIG. 6B shows a gel electrophoresis plate wherein lanes 4 and 5 correspond to eluant from a device with the thermal oxide+hydrogen peroxide/sulfuric acid clean. Again, no nucleic acid band is seen—suggesting that little or no reversible binding of DNA to the binding material was observed.

FIG. 6A, lanes 2 and 3 correspond to eluant from a device with the thermal oxide+plasma etching binding material. Bands indicate the presence of nucleic acid and the success of the such treated binding material to bind the nucleic acid. Shown in FIG. 6B, lanes 2 and 3, is the eluant from a device with thermal oxide+plasma etching+hydrogen peroxide/sulfuric acid cleaned binding material. As can be seen, nucleic acid is present and is indicative of reversible binding events between the nucleic acid analyte and the binding material.

Thus, it is apparent that thermal oxide-generated binding materials alone are, without further treatment, insufficient for utilization as binding materials for DNA of the 200 base pair fragment investigated in this experiment, according to the present invention. Plasma treatment has been shown to be a suitable treatment for activating the thermal oxide-generated binding material.

### EXAMPLE 3

This Example serves to illustrate the effect of temperature on the elution efficiency.

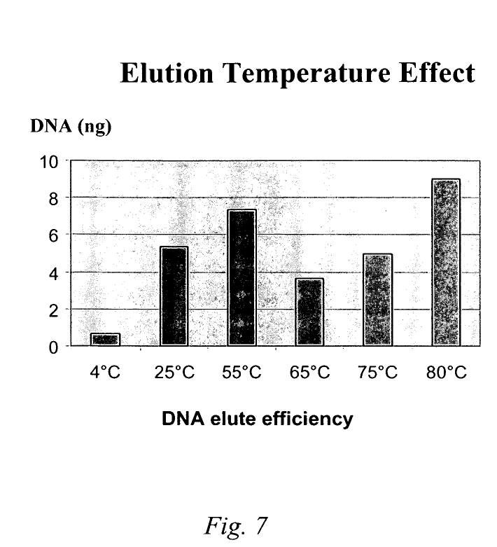

Experiments were performed on 1 cm×1 cm squares of silicon with thermal silicon oxide. The oxide surface underwent a CHF₃ plasma etching process with CHF₃ and O₂. Five μg of pure DNA was diluted in 8 μl of 6M guanidine hydrochloride solution. The DNA was then placed on the surface of a silicon die and a second silicon die was placed on top, forming a sandwich arrangement. The die were then placed in an airtight container with controlled humidity and incubated for 15 minutes. The die were then rinsed three times in fresh guanidine hydrolchloride (100 μl each time), followed by rinsing three times with 70% ethanol (100 μl each time). The samples were then allowed to dry at room temperature before elution was carried out. Wafers were eluted four times (total of 280 μl), each time with fresh 70 μl of 10×TE buffer (described earlier), and each time for 5 minutes, at the control temperatures between 4° C. and 80° C. as shown in FIG. 7. The amount of DNA eluted was quantified using the intercalating dye Picogreen™.

Referring to FIG. 7, the bar graph shows how the amount of DNA eluted varies with the temperature, showing a maximum around 55° C., as well as a steady increase from 65° C. to 80° C. For the experiments performed between 4° C. and 80° C., the maximum elution of DNA was achieved at 80° C. In general, an increase in temperature will result in increased diffusion and weakening of intermolecular bonds, and maximum elution efficiency should occur at higher temperatures. While not intending to be bound by theory, the peak around 55° C. in FIG. 7 is believed to be indicative of secondary mechanism

The results of the experiments performed on the above-described silicon die were applied to the micromachined binding reactor. The reactor was thermally isolated from the device substrate to allow for independent thermal operation. Resistive heaters and resistive temperature sensors were fabricated on the glass above the thermally isolated binding reactor, but not on the glass region that formed the cap for the micromachined reactor (ie., the heaters surrounded the reactor). This resulted in a binder where the temperature could be controlled electrically and independently from the substrate. The micromachined binding reactor was fabricated using the process sequence described in Example 1. The reactor was able to operate at 80° C. with the substrate connected to a thermal heat sink to ensure the rest of the system operated at room temperature, or at a temperature independent of the binder temperature.

### EXAMPLE 4

This Example serves to illustrate the binding efficacy of silane-deposited binding material with and without concurrent plasma treatment. It further compares these results (i.e., binding efficiency) with those of thermal oxide+plasma treatment binding material, and it relates all of these as a function of three temperatures.

Experiments were performed on 1 cm×1 cm squares of silicon with thermal silicon oxide and a silane based PECVD silicon oxide, deposited at 400° C. The thermal silicon oxide surface underwent a CHF₃ plasma etching process with CHF₃ and O₂ and the silane based PECVD oxide samples were divided into two sets, where one set also underwent a CHF₃ and O₂ etching process. Five μg of pure DNA was diluted in 8 μl of 6M guanidine hydrochloride solution. The DNA was then placed on the surface of a silicon die and a second silicon die (with oxide of the same type as the first) was placed on top, forming a sandwich arrangement. The die were then placed in an airtight container with controlled humidity and incubated for 15 minutes at different temperatures. The die were then rinsed three times in fresh guanidine hydrolchloride (100 μl each time) followed by rinsing three times with 70% ethanol (100 μl each time). The samples were then allowed to dry at room temperature before elution was carried out. Wafers were eluted three times (total of 210 μl), each time with fresh 70 μl of 10×TE buffer (described earlier). The first elution was for 20 minutes, the second for 15 minutes, the third for 5 minutes. All elutions were carried out at room temperature. The amount of DNA eluted was quantified using an intercalating dye Picogreen™.

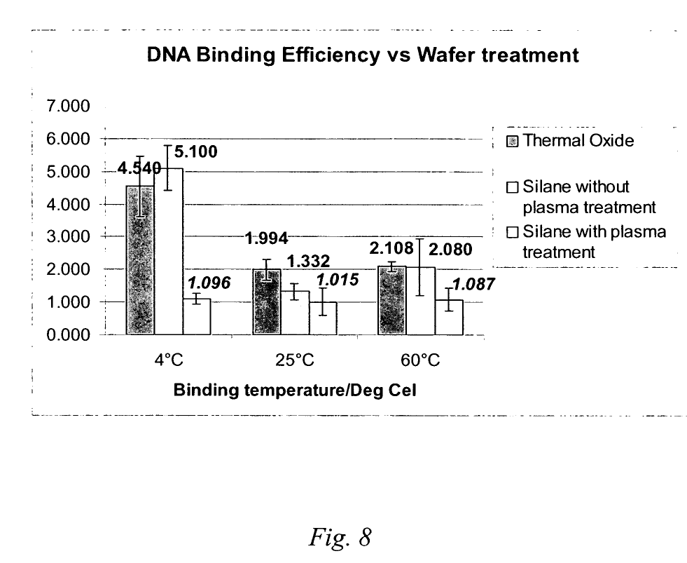

Referring to FIG. 8, wherein the x-axis is binding temperature in degrees Celsius, and the y-axis is nanograms (ng) of DNA eluted. The results show the amount of DNA eluted (in ng) for the three different binding temperatures. The maximum binding efficiency was achieved at 4° C. for the thermal silicon oxide and the silane based PECVD oxide without plasma etching treatment. The binding to silane based PECVD oxide that had undergone plasma etching was low under all conditions and not enhanced by changes in the binding temperature. These results were incorporated into the micromachined binder by cooling the system substrate to 4° C. through cooling of the heat sink thermally connected to the substrate.

All patents and publications referenced herein are hereby incorporated by reference. It will be understood that certain of the above-described structures, functions, and operations of the above-described embodiments are not necessary to practice the present invention and are included in the description simply for completeness of an exemplary embodiment or embodiments. In addition, it will be understood that specific structures, functions, and operations set forth in the above-described referenced patents and publications can be practiced in conjunction with the present invention, but they are not essential to its practice. It is therefore to be understood that the invention may be practiced otherwise than as specifically described without actually departing from the spirit and scope of the present invention as defined by the appended claims.

1. A device for microfluidic nucleic acid processing comprising:
a) a silicon substrate;
b) at least one inlet capable of providing for the introduction of microliter quantities of a material selected from the group consisting of mammalian blood, buffered saline solution, lysing agent, saline solution, alcohol, air, and combinations thereof;
c) a mixer, housed within a mixing chamber and capable of mixing blood with buffered saline solution;
d) a lysing chamber into which a lysing agent can be delivered for the purpose of lysing cellular membranes;
e) at least one outlet capable of removing material from the device;
f) a binder chamber comprising a binding material capable of binding nucleic acid under suitable conditions; and
g) at least one valve capable of directing flow through the device.

2. The device of claim 1, further comprising a filter, housed within a filter chamber and comprising a pore size sufficiently large enough to trap white blood cells, but sufficiently small so as to allow red blood cells to pass through.

3. The device of claim 1, wherein the device is monolithic in design and construction.

4. The device of claim 1, wherein the mixing chamber and the lysing chamber are the same.

5. The device of claim 2, wherein the lysing chamber and the filter chamber are the same.

6. The device of claim 1, further comprising at least one optically-accessible channel.

7. The device of claim 1, further comprising bond pads for making electrical connections.

8. The device of claim 1, further comprising a glass wafer cover.

9. The device of claim 1, further comprising a heating means for selectively heating regions of the device.

10. A nucleic acid binding material made by a process comprising the steps of:
a) providing a silicon substrate;
b) treating the silicon substrate with a thermal oxide process to provide for a silicon oxide surface; and
c) plasma etching the silicon oxide surface with a plasma etchant process.

11. The nucleic acid binding material of claim 10, wherein the plasma etchant process comprises a combination of CHF₃ and 02 plasma etching treatments.

12. A nucleic acid binding material made by a process comprising the steps of:
a) providing a substrate; and
b) depositing silane-based silicon oxide onto the substrate using a plasma-enhanced chemical vapor deposition process.

13. The nucleic acid binding material of claim 12, wherein the substrate comprises silicon.

14. A method for processing nucleic acid comprising the steps of:
a) mixing microliter quantities of mammalian blood and saline solution in a mixing chamber to produce a diluted blood mixture;
b) flowing the diluted blood mixture and a lysing agent into a reaction chamber, wherein the lysing agent ruptures the cellular walls and liberates nucleic acid contained within white blood cells; and
c) flowing the mixture comprising the liberated nucleic acid through a binding chamber, wherein said binding chamber comprises a binding material that selectively binds nucleic acid under conditions of high salt content.

15. The method of claim 14, further comprising a step of filtering the diluted blood mixture through a filter to separate nucleic acid-containing white blood cells from red blood cells.

16. The method of claim 14, further comprising a step of rinsing the nucleic acid bound to the binding material with a rinsing agent.

17. The method of claim 16, wherein the rinsing agent is ethanol.

18. The method of claim 14, further comprising a step of drying the nucleic acid bound to the binding material with a drying method.

19. The method of claim 18, wherein the drying method is force convection.

20. The method of claim 18, wherein the drying method comprises a flow of air.

21. The method of claim 14, further comprising a step of treating the nucleic acid bound to the binding material with a low-salt solution to free the nucleic acid from the binding material and enable collection of it.

22. A monolithic device for microfluidic nucleic acid processing comprising:
a) a means for introducing cellular material into the device;
b) a means for introducing a lysing agent into the device;
c) a means for mixing the cellular material with the lysing agent so as to effect the rupture of the cell membranes and liberate the cellular components;
d) a means for selectively binding nucleic acid;
e) a means for rinsing the bound nucleic acid;
f) a means for drying the bound nucleic acid; and
g) a means for de-binding and eluting the nucleic acid.

23. The device of claim 22, wherein the cellular material and the lysing agent are introduced via inlets.

24. The device of claim 22, wherein the means for mixing the cellular material with the lysing agent comprises a coiled capillary micromixer.

25. The device of claim 22, wherein the means for selectively binding nucleic acid comprises the binder material of claim 10.

26. The device of claim 22, wherein the means for selectively binding nucleic acid comprises the binder material of claim 12.

27. The device of claim 22, wherein the means for selectively binding nucleic acid comprises the use of a high-salt solution.

28. The device of claim 22, wherein the means for drying the bound nucleic acid comprises force convection drying.

29. The device of claim 22, wherein the means for de-binding and eluting the nucleic acid comprises rinsing with a low-salt solution.

30. The device of claim 22, wherein the means for de-binding and eluting the nucleic acid comprises rinsing with a high-purity water
